(19) United States
(12) Patent Application Publication
(10) Pub. No.: US 2012/0271192 A1
(43) Pub. Date: Oct. 25, 2012

(54) METHOD AND APPARATUS FOR ANALYSING THE STRUCTURE OF BONE TISSUE

(75) Inventors: Marcin Just, Wroclaw (PL); Przemek Los, Warsaw (PL)

(73) Assignee: CarieScan Limited, Dundee (GB)

(21) Appl. No.: 12/594,262

(22) PCT Filed: Apr. 2, 2008
(86) PCT No.: PCT/GB2008/001147

§ 371 Date: Mar. 18, 2010

(30) Foreign Application Priority Data
Apr. 2, 2007 (GB) 0706439.7

(51) Int. Cl. A61B 5/053

(52) U.S. Cl. 600/547

(57) ABSTRACT

A method of analysing the structure of bone tissue comprises placing first and second electrodes in electrical contact with the bone tissue to be analysed such that the bone tissue forms at least part of an electrical circuit between the first and second electrodes. Alternating electrical signals are applied to the circuit and the electrical response of the circuit is monitored. The monitored response is processed to generate output data representative of the structure of the bone tissue.

### FIELD OF THE INVENTION

The present invention relates to a method and apparatus for analysing the structure of bone tissue.

### BACKGROUND TO THE INVENTION

A number of techniques are known in the art for analysing the structure of bone tissue. The analysis is desirable in most applications to be performed in vivo whilst the bone tissue in question is present within the body of a living subject. The most widely used techniques are X-ray based which typically involve the imaging of the bone tissue and are performed by exposing the subject to a beam of X-rays and recording the resultant absorption image data. Such techniques have been used extensively and successfully for many years offering information on the bone geometry and, to some extent, its substructure geometry together with some information of general bone density.

Another useful technique is that of magnetic resonance imaging (MRI) which can provide very detailed information upon the internal structure of the bone tissue. The X-ray and MRI techniques are required to be performed by trained staff and involve expensive hardware, normally meaning that these systems are only available in well equipped laboratories and hospitals. Another technique uses ultrasound which is significantly less expensive although it has found little practical application, due in part to the “noisy” data which results.

In addition to the practical techniques described above, a small number of academic studies have been undertaken using electrical impedance tomography (EIT). For example it has been shown that rudimentary “images” based upon resistivity can be generated using an electrode array. A recent study (Sierpowska, J., et al., Effect of human trabecular bone composition on its electrical properties, Medical Engineering & Physics, 2006) found some correlation between electrical parameters and the composition of bones.

Whilst there have been some academic studies regarding the use of electrical impedance in obtaining information about bone tissue, none have provided an effective and reliable technique for assessing the structure of bone. There is therefore a need to improve electrical impedance methods if they are to be used in practical and commercial applications.

### SUMMARY OF THE INVENTION

In accordance with a first aspect of the present invention we provide a method of analysing the structure of bone tissue comprising:

a) placing first and second electrodes in electrical contact with the bone tissue to be analysed such that the bone tissue forms at least part of an electrical circuit between the first and second electrodes;

b) applying an alternating electrical signal to the circuit and monitoring the electrical response of the circuit; and,

c) processing the monitored response to generate output data representative of the structure of the bone tissue.

The present inventors have overcome the problems encountered by the early studies in this field and, in accordance with the present invention, now provide a technique for providing reliable data regarding the structure of bone tissue obtained using electrical signals.

It has been found extremely advantageous to use the first and second electrodes to not only apply the alternating electrical signal to the circuit but also to monitor the electrical response of the circuit. This produces excellent results and greatly simplifies the four electrode techniques used in related electrical impedance monitoring fields. The use of only two electrodes provides benefits in terms of cost and also in reducing the number of possible errors which can be introduced during the practical performance of the method. With two electrodes the measurement set-up is much simpler and limited to the choice of positioning of just two electrodes. It is extremely advantageous for a commercial application of the method and it may simplify the theoretical models used to interpret the measurements. In such cases the measurement not only includes the tissue between the electrodes but additionally the interfacial region at each electrode surface. Theoretical finite element model (FEM) analysis shows that in the case of injecting current, the highest potential differences are observed at the electrode interfaces and consequently the accuracy is highest for measurements at these contact points. A two electrode system makes the operation of a practical device much simpler and reliable. There is also a general prejudice within the field of electrical impedance monitoring towards using a four electrode technique. In a four electrode system it is generally accepted in bio-impedance studies that the impact of an unknown contact impedance is limited by separating the two functions of the electrodes. In this way, two discrete electrode pairs are used: the first for passing current (called a drive pair) and a second for measuring the boundary voltage (receiving pair). Using high input impedance equipment the current flow at the receiving electrodes is negligible and consequently voltage measurement error is minimised. In addition, the voltage electrodes can be placed away from disturbances localised at the sites where the current is injected which can improve the accuracy of ac impedance measurements.

The invention can be implemented using a single carefully selected applied signal frequency. Preferably, however, multiple frequencies may be used and these may be applied either simultaneously or in a serial manner. One aspect of the invention is the realization that using relatively low frequencies provide advantageous results. Typically in other electrical bioimpedance fields, the signals are applied at frequencies of at least tens and typical hundreds or even thousands of kHz. In the present application it is preferred to use one or more applied signals at frequencies less than 20 kHz. Greater advantage is provided by including one or more applied frequencies of the alternating signal at less than 1 kHz and most preferably at 200 Hz or less. In summary, it has been found that lower frequencies than those conventionally used in electrical impedance measurements should be used since these provide a greater distinction between specific types of bone structure. The applied signals are generally of an alternating form that may be applied as a controlled alternating current or voltage. Typically the signal alternates periodically as a sinusoidal function although other waveforms can be used.

The term “structure” used herein is intended to include all aspects of the bone structure that influence the electrical impedance properties. These include not only the physical structure (and substructure) of the bone tissue itself, but also its composition. In particular, the structure is intended to include the mineral content of the bone tissue, including its calcium content and other materials that influence the tissue's electrical properties. It is known that bone is a relatively hard and lightweight material, formed mostly of calcium phosphate in the form calcium hydroxyapatite. Bone tissue generally takes two forms, these being compact and cancellous. Typically the outer layer of bone tissue is compact and accounts for about 80% of the bone mass in an adult human. The cancellous bone is known as “trabecular” in structure in that it has an open, meshwork or sponge-like structure. This accounts for about 20% of the total bone mass. Although the bone structure is therefore rather complex, in a like-for-like comparison between similar bones in different subjects, the electrical properties in particular are strongly dependent upon the mineral content. Thus the structure of the bone tissue and its electrical impedance properties have a strong dependence upon the bone mineral density.

As discussed above, typically the invention is performed in vivo upon live subjects. Such subjects are typically human although alternatively, the bone tissue structure of other animal species may also be assessed using the invention.

Although preferably a pair of electrodes is used to perform each of the functions of applying the signal to the circuit, together with monitoring the circuit response, a four electrode system can be used in which two electrodes are exclusively used to apply the signal and another two are used to monitor the response.

In the case of a two electrode system, typically the first electrode is placed at or adjacent to a first surface of the bone tissue and the second electrode is placed at or adjacent to a second surface of bone tissue. In most cases the bone will be covered by other body tissue, including muscle, fat and cartilage although it has been found that these do not significantly affect the results. In fact the most significant other influence upon the circuit is the interface between skin and the electrode surface.

In principle the invention may be applied during a surgical procedure such that one electrode or both electrodes may be applied directly to the bone itself. This may also apply where the method is used in an in vitro situation. However, typically the first and second electrodes are each placed at surfaces which are adjacent to the respective surfaces of the bone tissue, although separated somewhat therefrom. Thus preferably the first and second electrodes are positioned at locations on the skin surface where the corresponding bone or bones whose tissue is to be analysed lie close to the surface of the skin. Preferably an electrically conductive material such as conductive gel is applied to each electrode and/or the skin surface against which the particular electrode is placed. Following application of the conductive material, it is preferred to wait for a short period whilst the material permeates into the skin somewhat and improves the electrical contact between the skin and the electrode in question. Alternatively, gel-based electrodes can be used that encapsulate an aqueous electrolyte in a suitable polymer matrix.

In accordance with electrical impedance theory, the monitored response of the circuit is determined by the input signal and a notional equivalent circuit representative of the physical properties of the material being analysed. Preferably the method of processing the response comprises deriving one or more electrical characteristics from the monitored response and then comparing these characteristics with a structure characteristic of the bone tissue in accordance with a predetermined relationship. For example, the one or more electrical characteristics may include the applied electrical stimulus, the acquired electrical response, and the phase shift between them. It has been found that a relatively simple relationship between the electrical characteristics can then be used to generate a further electrical characteristic such impedance, conductance or permittivity which can then be related to the structure characteristic. The structure characteristic is preferably a measure of bone mineral density (BMD) which is a well studied bone structure parameter. The preferred method of measuring such a bone structure is by the use of dual energy X-ray absorptiometry (DXA or DEXA). This uses two X-ray beams of different energies so as to determine a numerical value relating to the bone density of the bone in question. This is expressed as a numerical BMD value which can then be used to generate further values that compare with young normal subjects (T-score) and those of a similarly aged population (Z-score).

The predetermined relationship between the electrical characteristics and the structured characteristic of the bone tissue (which may include more than one structure characteristic), may include an analytical approach, an empirical model, a neural network or other statistically derived model.

The present method provides many benefits over the prior art methods in terms of its accuracy and practical costs. It is voltage and current limited to significantly within medical safety restrictions. This means that concerns over safety are avoided compared with repeated use of harmful ionizing X-ray radiation.

The electrical signals may be applied to a number of regions of the human body, these including a hip region, a heel region or a spine region (particular vertebrae L1 to L4). Preferably, however, the method is applied to a forearm region of a subject human body. In this case, one of the first and second electrodes is preferably positioned against the styloid process of the radius or ulna and the other of the first and second electrodes positioned adjacent to the olecranon process. These particular locations provide a good length of bone tissue (radius or ulna bones) through which the electrical current is passed, and they also provide points of close approach to the skin of the heads of these bones. A further advantage is that a great deal of data is available from other techniques relating to these regions of the body, particularly DXA techniques.

In accordance with a second aspect of the present invention we provide apparatus for analysing the structure of bone tissue comprising:

a first and second electrode in electrical contact with the bone tissue to be analysed such that the bone tissue forms at least part of an electrical circuit between the first and second electrodes;

a signal generator adapted to apply an alternating electrical signal to the circuit through the first and second electrodes;

a monitoring device for monitoring the electrical response of the circuit; and,

a processor adapted when in use to process the response monitored by the monitoring device and to generate output data representative of the structure of the bone tissue.

Thus the apparatus in accordance with the second aspect may be used in the performance of the method according to the first aspect.

The signal generator used to apply the alternating electrical current is adapted to generate electrical signals at one or more than one frequency. Where a plurality of frequencies are to be applied, these are preferably applied in a serial manner. Multiple frequencies may be applied simultaneously and its time domain response transformed to the frequency domain.

Typically the signal generator and the monitoring device are formed within a single unit. The apparatus is preferably portable and may be handheld. Two units in the form of a base station and a handheld unit for example can be used, there being in bidirectional communication, preferably wirelessly. Typically the electrodes are formed from an electrically conductive material (these including biocompatible metals such as stainless steel). In principle other conductive biocompatible materials such as conductive polymers could be used as an alternative. One of the first or second electrodes is preferably formed having an electrically conductive contact surface containing a depression suitable to receive the elbow (olecranon) of a human subject. Alternatively a flexible gel-based electrode can be used that conforms to the surface applied to.

As will be appreciated, the structure of the bone tissue derived as a result of the use of the present invention can be used to detect the presence, progression or regression of osteoporotic or osteopenic bone tissue. Other medical disorders which are indicated by bone structure may also be detected.

In accordance with a third aspect of the present invention we provide a method of monitoring changes in the structure of bone tissue within a subject body, comprising:

analysing the structure of the bone tissue of a particular subject at different times using a method according to the first aspect of the invention;

comparing the bone structure represented by the output data with bone structure representative of the type of subject monitored; and,

selectively applying a treatment to subject body as a result of the comparison.

Thus we provide a method of screening for particular conditions including osteoporosis and osteopenia. Preferably the analysis times are separated by at least one year, more preferably a period that is clinically acceptable. The comparison may be performed in accordance with different types or groups of human subjects, this taking into account one or more parameters such as the sex, age, size, weight, medical conditions and drug treatment histories of the subjects.

Preferably the treatment is applied and when the analysed subject has a represented bone structure which differs from the representative structure for the subject by more than a predetermined magnitude. This may be related to a Z-score or T-score.

### BRIEF DESCRIPTION OF THE DRAWINGS

An example of method and related apparatus for performing the invention are now described with reference to the accompanying drawings, in which:

FIG. 1 shows a schematic representation of apparatus according to the example for implementing the method;

FIG. 2 shows the application of the first and second electrodes to a human forearm;

FIG. 3 shows a flow diagram in accordance with the operation of the example method;

FIG. 4 shows the measurement performance of an example device against a benchmark Solartron frequency response analyser: (a) Bode plots and (b) Nyquist plot;

FIG. 5 is a table showing measured BMD data for a number of test subjects; and,

FIG. 6 is a table showing measured impedance data for five subjects selected from the table of FIG. 5;

FIG. 7 shows the relationship between phase shift and the magnitude of impedance for selected BMD values;

FIG. 8 shows the relationship between magnitude of impedance at minimum phase and BMD values;

FIG. 9 Shows an example of diagnostic accuracy for osteopenia (using DEXA BMD-U33 as reference) analysed by a statistical method.

FIG. 10 Shows an example of diagnostic accuracy for osteoporosis (using DEXA BMD-U33 as reference) analysed by a statistical method.

### DESCRIPTION OF MAIN EXAMPLE

We now describe a suitable apparatus for implementing the invention together with associated method steps. This example is described in the context of analysing the structure of human bone tissue with a view to obtaining information relating to the bone density. This information can then be used as some of the information upon which to base a medical diagnosis relating in particular to osteopenia or osteoporosis.

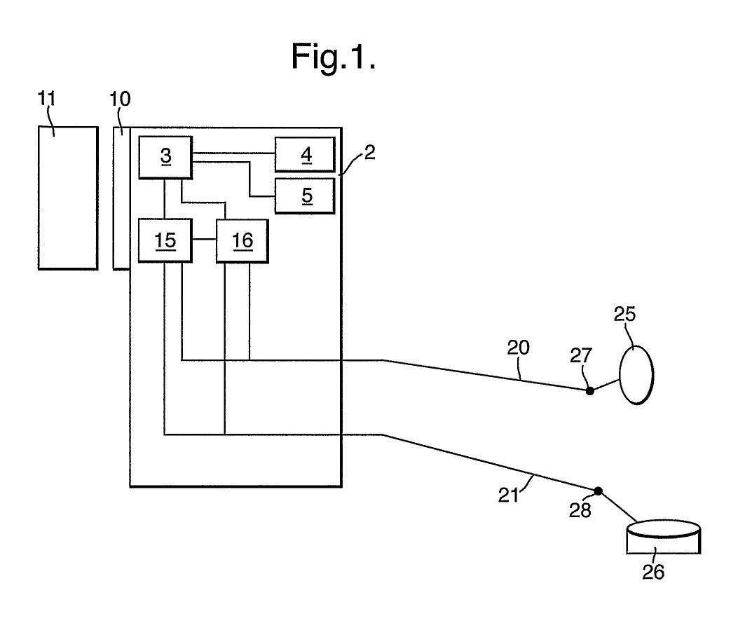

Referring now to FIG. 1, an apparatus for monitoring changes in the structure of bone is illustrated and generally indicated at 1. The apparatus comprises a portable unit 2 which may be either handheld or of larger dimensions such as those of a laptop computer. The portable unit 2 contains an internal computer 3 comprising a processor and other associated devices including a memory for storage of data and a device controller. The computer 3 is in communication with a display 4 and an input device such as a keypad 5 allowing an operator to issue commands to the portable unit 2. The portable unit 2 is powered by an internal rechargeable power source 10 which can be charged by an external inductive charger or medically approved power supply unit indicated at 11.

The internal device controller within the computer 3 is arranged in communication with a signal generator 15 or output stage typically comprising a direct digital synthesizer (DDS) or digital to analogue converter (DAC). This enables multiple frequency waveform generation which is capable of providing electrical signals to corresponding output ports under the control of the computer 3. Computer 3 may therefore control the amplitude, phase and frequency of the signals produced by the signal generator 15. In the present example the signal generator only issues a single frequency signal at any one time although in an alternative example the embedded software can define a multi-frequency waveform. The analogue circuit supplies the signals to two ports on the external surface of the portable unit 2 (not shown). Into these ports corresponding leads 20 and 21 are removably coupled which carry the generated signals between the unit 2 and first and second respective electrodes 25 and 26. Alternatively, the connection points for the electrodes may be integrated directly into the enclosure of the device.

The first and second electrodes 25 and 26 are also removably coupled by simple connectors to the distal ends of each of the leads 20, 21. This allows the electrodes 25, 26 to be disposable for the purposes of sterility. Preferably the electrodes are therefore used only upon a single subject. The electrodes 25, 26 are used to pass the generated current through the body of a subject 50 in a manner to be described later. The presence of the subject body 50 between the electrodes 25 and 26 completes the electrical circuit with the signal generator 15. It is the response of this electrical circuit to the applied signals that provides information concerning the bone structure of the subject body 50.

In order to measure the response, a monitoring device 16 or input stage, typically comprising an analogue to digital converter (ADC), is provided within the portable unit 2, this being in communication with each of the signal generator 15 and computer 3. It will be appreciated that the signal generator 15 and monitoring device 16 may be formed as a single signal generation and analyser unit (analogue circuit). The monitoring device 16 is also connected internally to the circuit formed by the signal generator 15 and electrodes 25, 26.

Depending upon the particular implementation, the signal generator/monitoring device may take the form of a lock-in amplifier, frequency response analyser, or a fast fourier transform device (in the event that simultaneous multiple frequencies are produced).

When in typical use in association with the present example, the signal generator 15, under the control of a computer 3 produces electrical signals in the form of sinusoidal alternating current flow within the circuit produced by contact between the first and second electrodes and the particular part of the subject body in question, this current being of a magnitude sufficiently low to comply with all international medical safety standards (IEC60601-1 and its deviations). In the present case the computer 3 monitors the output current of the signal generator 15 by communication with the signal generator.

The monitoring device 16 measures the potential difference between the two parts of the circuit formed, therefore effectively between the first and second electrodes 25, 26. Of course this measured electrical response of the circuit also has an alternating frequency, although this is not necessarily the same as the applied frequency. As will be appreciated, a phase shift typically exists between the applied voltage and the response current. The monitored response from the monitoring device 16 is also provided to the computer 3 which uses this data to calculate an impedance characteristic of the circuit formed and then goes on to calculate the corresponding bone tissue structure in accordance with an internal model, also to be described further later.

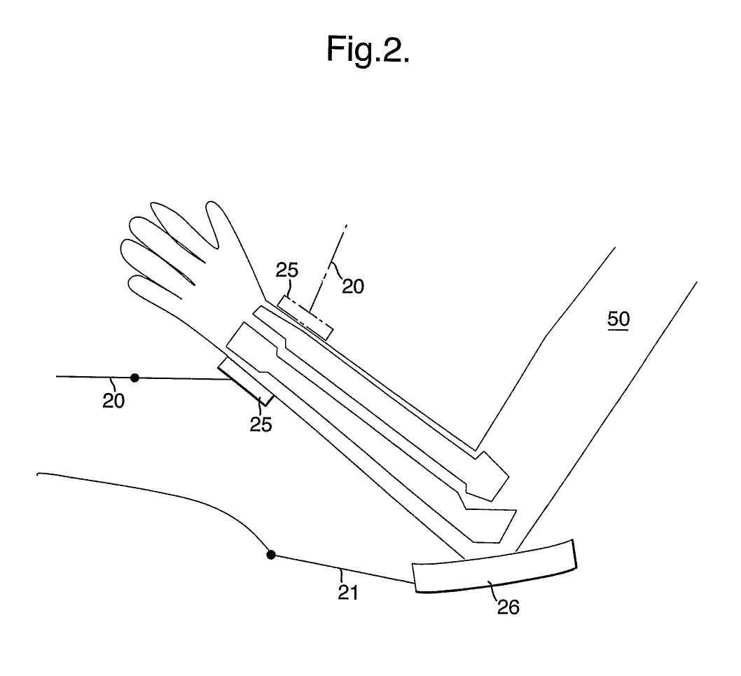

FIG. 2 shows the use of the electrodes 25 and 26 in applying the generated electrical signals to the forearm of a subject body 50. As can be seen from FIG. 2, the first electrode 25 is applied manually to the wrist as of a subject body 50 where it can be placed adjacent to the heads of either of the arm bones (either caput ulnea or head of the radial bone). Electrical contact between the electrode and the skin is achieved by applying an electrically conductive gel on the surface of the electrodes. Typically this is then held against the skin where the conductive gel permeates into the upper skin layers. A typical material for production of the first electrode is stainless steel or another conductive biocompatible electrical conductor.

The second electrode 26 is again also formed with a conducting material such as stainless steel and is placed on a stable base such as the upper surface of a table. The electrode is slightly hollowed and has dimensions of approximately 30 mm in diameter and a depth of hollow of 5 mm. Around the periphery of the electrode is provided a non conductive surround. Again as for the first electrode, a small amount of gel is placed in the hollow of the electrode and the elbow (olecranon) is placed inside the hollow of the second electrode. Typically the angle between the forearm (lower) and the upper arm is about 100 degrees and the open palm of the subject is placed vertically. In FIG. 2 the two possible positions of the first electrode 25 are illustrated placed adjacent the ulna bone (little finger side) or the radius bone (thumb side) of the forearm. The positioning of the second electrode 26 below the elbow is also illustrated.

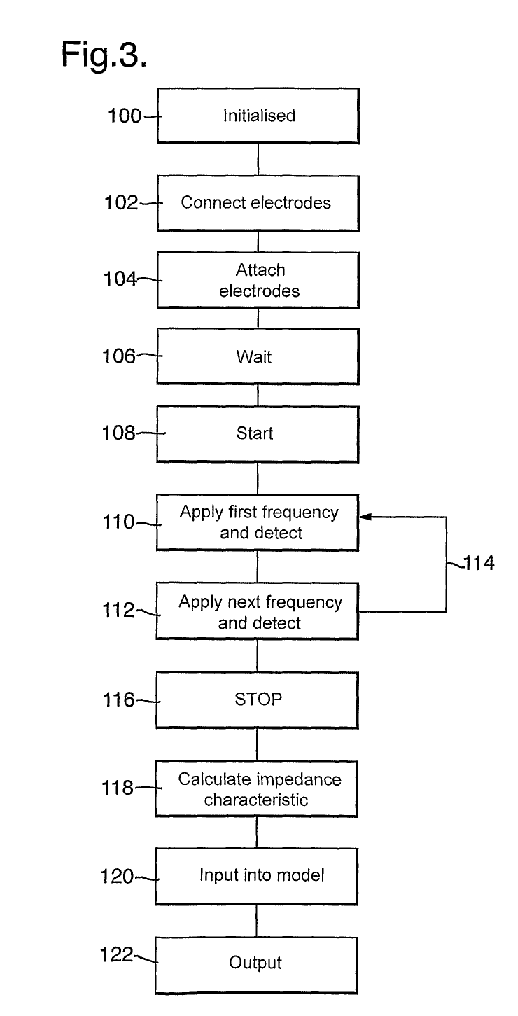

Turning now to FIG. 3 an example method is described for implementing the invention using the apparatus described above.

At step 100 the unit 2 is initialised by inputting data including the details of the subject's identity, together with the part of the subject body 50 which is to be used in the measurement, such as the ulna or radius in the forearm of a subject body 50.

As a result of the input of such data by the user, for example using the input device 5, the computer system selects from a lookup table or other stored data, the frequencies and currents which will be applied to the particular subject through the electrodes 25, 26. Once the system is initialised then at step 102, new sterile electrodes 25 and 26 are connected using their connectors 27, 28 to the supply leads 20, 21. A new pair of electrodes is provided for each new patient although if the patient has been subject to previous tests recently, then electrodes specific to that patient may be reused if sterliisation or disinfection is possible.

At step 104, the second electrode 26 is placed upon the upper surface of a table or other support structure, this electrode having the depression therein facing upwards. For solid electrodes such as stainless-steel, an electrically conductive gel is applied within the depression and a similar gel is applied to the contact surface of the first electrode 25. Alternatively, gel-based electrodes can be used. It must also be appreciated that device enclosure can be designed to be free-standing system that incorporates appropriate supports for each electrode. The forearm of the subject body 50 is then positioned such that their elbow (olecranon) is resting within the depression of the second electrode 26 whereas the first electrode 25 is then positioned pressed against the head of either the radius or ulna bones in either of the two positions shown in FIG. 2.

At step 106 a short wait is performed whilst the gel works into the surface of the skin so as to produce a good electrical contact. This may be aided by applying additional conductive gel directly to the skin in the region of contact with the electrodes 25, 26. Thereafter at step 108, a measurement sequence of method steps is begun. At step 110 a first frequency of electrical signals generated by the signal generator 15 is applied to the electrodes. The signals are at a first frequency which may be typically 100 Hz. This is performed for a short time period (approximately 1 second), during which the electrical response of the circuit so formed is recorded using the monitoring device 16. The data describing the output of the signal generator, together with the monitored response and their relative phase, are provided to the computer 3 during this step. At step 112 a second frequency, such as 200 Hz is then applied and again the monitoring is performed as in step 110. This is repeated at each of the frequencies selected by the computer 3 during the initialisation step 100.

In the present example about 20 frequencies are chosen between 100 Hz and 20 kHz and each frequency is applied in steps 110, 112 and 114 so as to provide a set of data spanning the range of frequencies. Alternatively, the steps 110, 112 and 114 may be replaced by a single output and subsequent acquisition of a waveform comprising the overlapping frequencies defined in initialisation step 100. At step 116 the process is stopped and the electrodes are removed from the subject body 50.

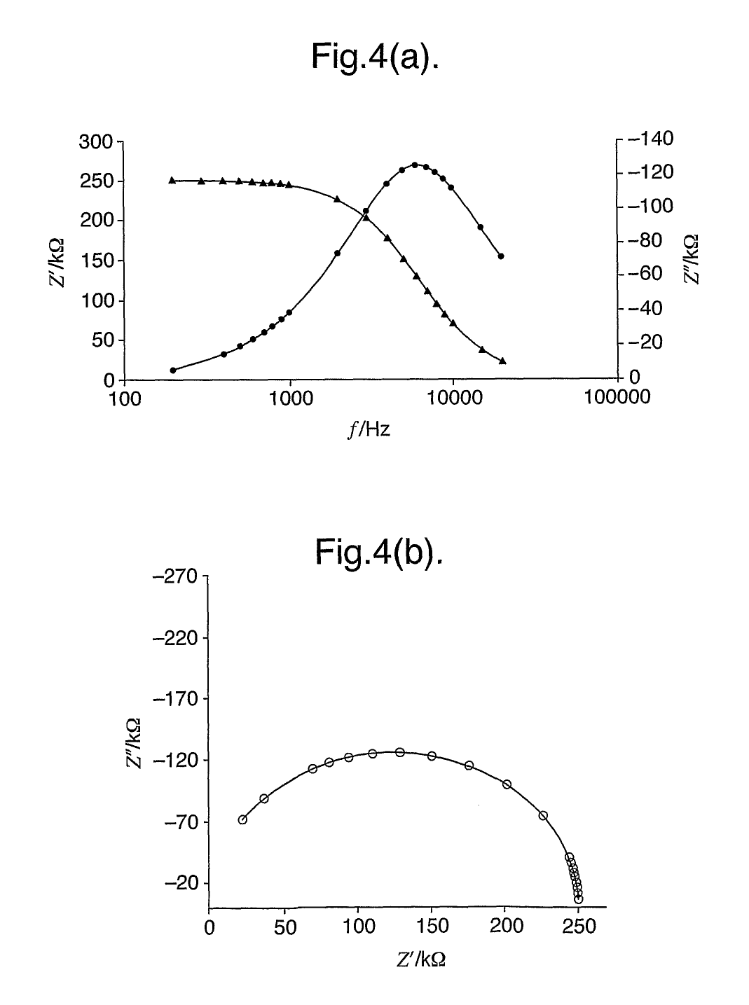

At 118 the processing steps performed by the computer begin. At step 118 in particular the applied voltage data and the monitored current data are converted into an electrical impedance characteristic. In the present case this is simply achieved by the division of the root mean square (rms) amplitude of the voltage monitored by the rms amplitude of the current supplied for each of the frequencies applied in steps 110 to 114. This gives an rms amplitude of impedance Z for each applied frequency. In addition to this data, the computer compares the applied current and voltage phases so as to determine the phase shift between the applied and the response signals. Thus the impedance characteristics of the rms impedance and the phase angle φ are determined for each frequency. In the present example the measurement performance was benchmarked against a calibrated Solartron® frequency response analyzer (FRA) 1260. The measured dummy cells were prepared from resistors and a combination of resistors in parallel with capacitors, where the resistive part values range from 47 kΩ to 10 MΩ. An example of the impedance response for a 250 kΩ∥100 pF cell is shown in FIG. 4. The device in the present example is represented by the point and the benchmark FRA is denoted by the solid line.

At step 120 the impedance characteristics are processed by the computer 3 by inputting them into a model which relates impedance characteristics with bone density values. In the present application of screening or monitoring osteoporosis and osteopenia, the bone density is measured as “BMD” values, these being values relating to measurements of bone density using DEXA as the accepted reference standard.

There are various models which may be used in step 120 to relate the measured impedance response with the BMD values. These include analytical models, although due to the complexities of biological systems, it has been found that simple empirical models provide good results in practical situations. Further, neural network analysis or other techniques may be used to derive such models.

When each of the impedance characteristics as a function of frequency is input into the model at step 122 one or more output data are obtained describing the bone characteristic in question. For example the output data may be in the form of an equivalent BMD value.

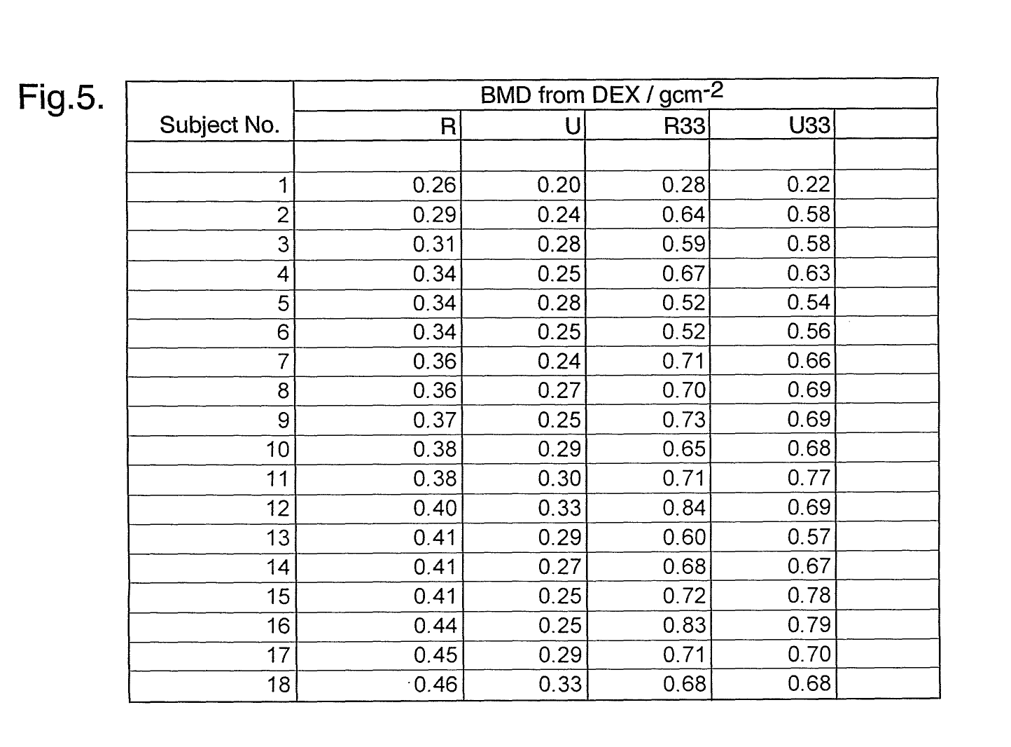

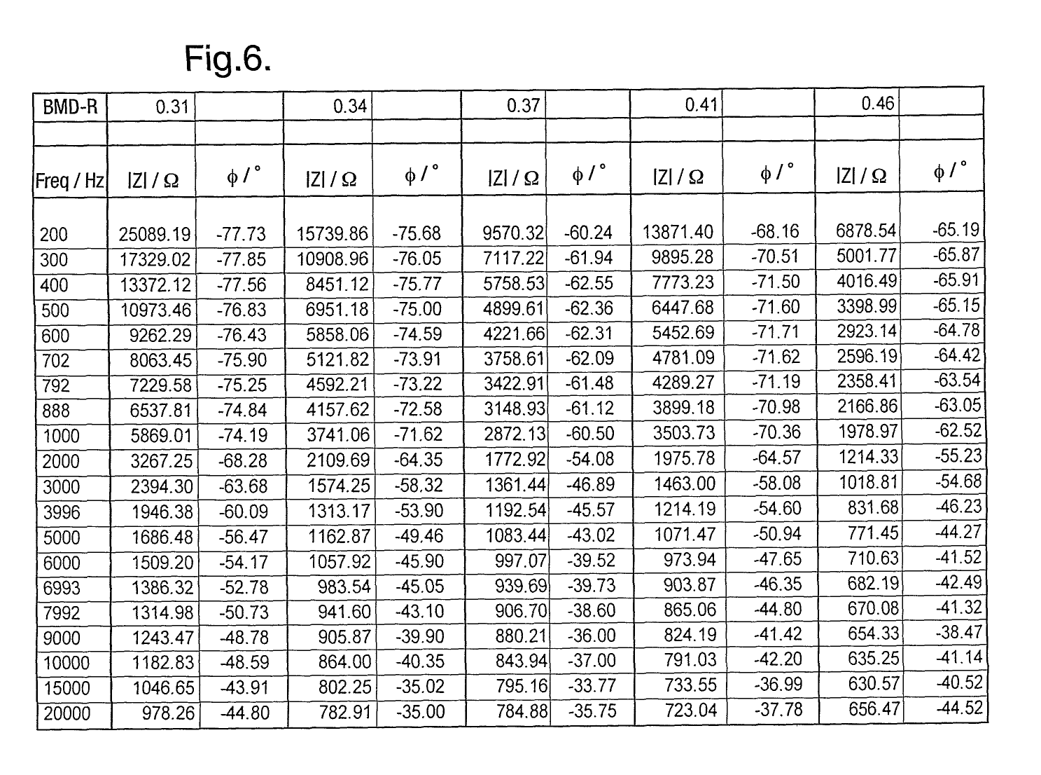

FIG. 5 shows a practical example of BMD values taken from both the radius and ulna of patients using DEXA analysis techniques. The BMD-R value is the bone mineral density value for the radius bone of the particular subject, the BMD-U value is taken for the ulna bone. In accordance with a second method of measurement, values for one third of the distance from the elbow to the head of the bone in each case illustrated as R33 and U33 are also shown in FIG. 5. Taking subject numbers 3, 4, 9, 15 and 18 as examples, the relevant frequency dependent values for the rms impedance and phase shift are shown in FIG. 6.

The modulus of impedance (effectively the rms impedance characteristic) drops significantly as the frequency increases from 200 Hz to 20 kHz. Furthermore the phase shift between the applied and measured signal also tends to reduce as the frequency increases. In comparing the subjects having a high BMD value with those of a low BMD value it can be seen that those with a high BMD value typically have a lower monitored impedance value than those with a lower BMD value. This is in line with reports in the published literature indicating that conductivity decreases as bone mineral density decreases. At a frequency of 20 kHz this difference is approximately 20% in the magnitude of the impedance whereas at 200 Hz the difference is approximately 260%. Thus from the above it can be concluded that very low frequencies of the order of a few hundred hertz are beneficial for determining BMD via a model from measured electrical impedance characteristics.

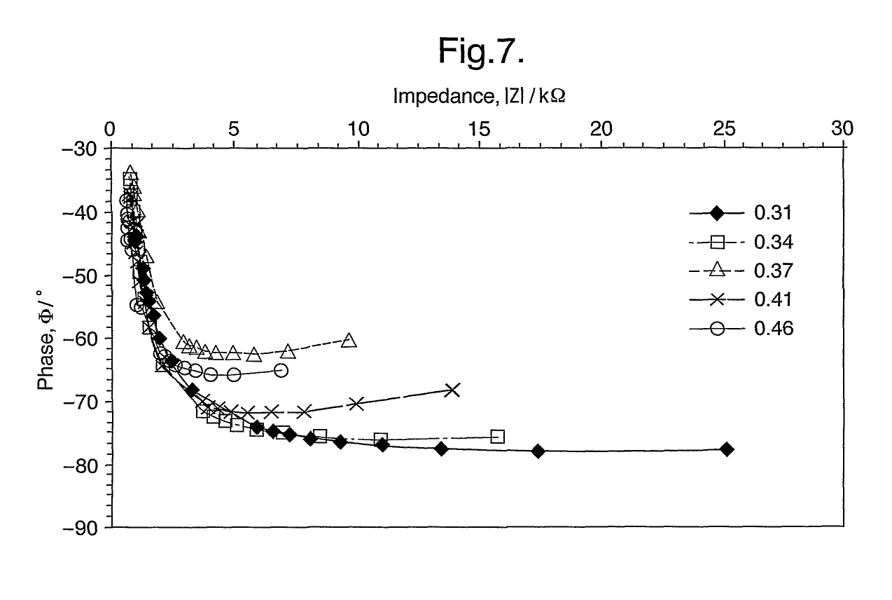

FIG. 7 shows the phase shift versus the modulus of the impedance value for the five subjects mentioned above. This shows a trend between the Bode graph shape (phase shift against modulus of impedance) and the BMD values for the radius bone of different subjects taken using DEXA measurements. This correlation can be observed between the DEXA obtained BMD parameters and other representations of the ac impedance graphs (including Nyquist, alternative Bode, admittance, and so on). This clear correlation demonstrates, even with the relatively simple methods described above, the significant link between the bone structure in terms of BMD values and the measured electrical impedance, particularly at relatively low frequencies.

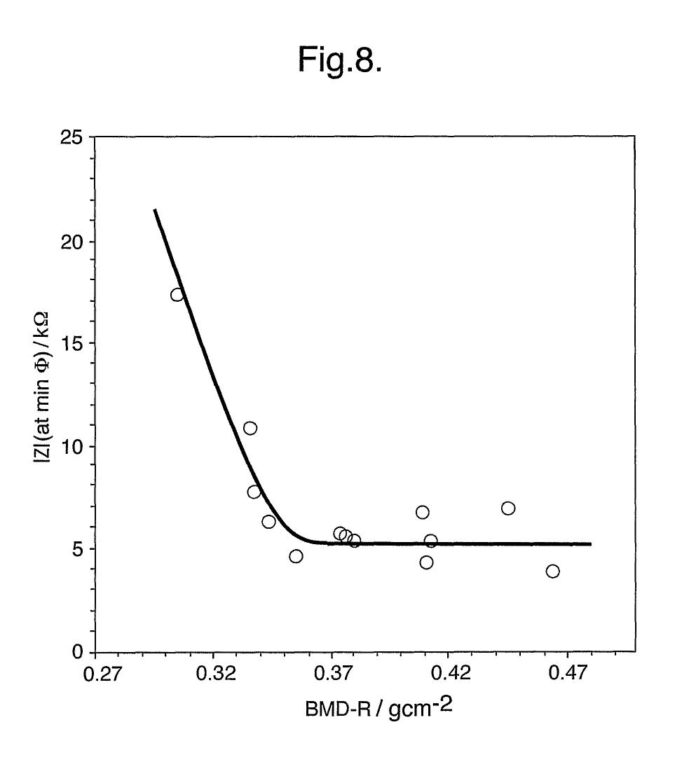

FIG. 8 shows an clear relationship between the measured data and the BMD values for the radius bone in each of the subjects. Here, using the table in FIG. 6, the minimum value in phase is used to obtain the equivalent magnitude of impedance value which is then plotted against the BMD R parameter.

The graph presented in FIG. 8 demonstrates two regions. A first region above approximately 0.37 BMD R is independent of the impedance and a second region showing strong dependency on impedance for values below approximately 0.37 BMD R. This can be associated to the clinical threshold values relating BMD parameters to conditions such as osteopenia and osteoporosis. Typical R value for sound bones is about 0.38 BMD, whereas osteopenia can be said to exist with a BMD R value lower than 0.32. Osteoporosis is considered to exist at R values lower than approximately 0.28.

Thus it can be seen that for values of BMD R around 0.37, there is an increase in the magnitude of the impedance taken at the minimum phase difference. Typically this occurs within the frequency range of below 1000 Hz. In FIG. 8 the data are plotted as a scatter graph and a trend line is overlayed. It will be appreciated that such a trend line or fit can be used as a basic model for correlating the impedance at the lowest phase angle with the BMD R value.

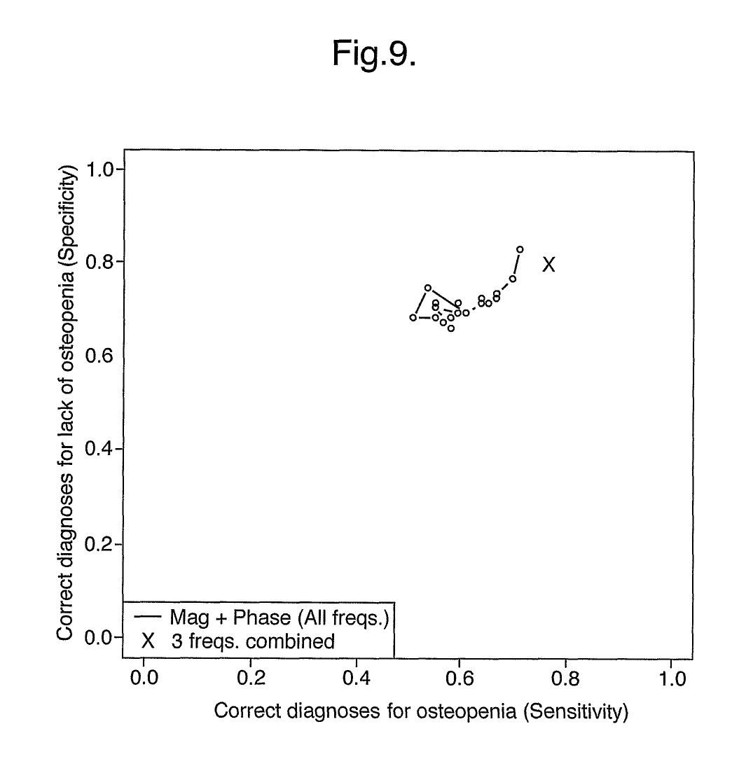

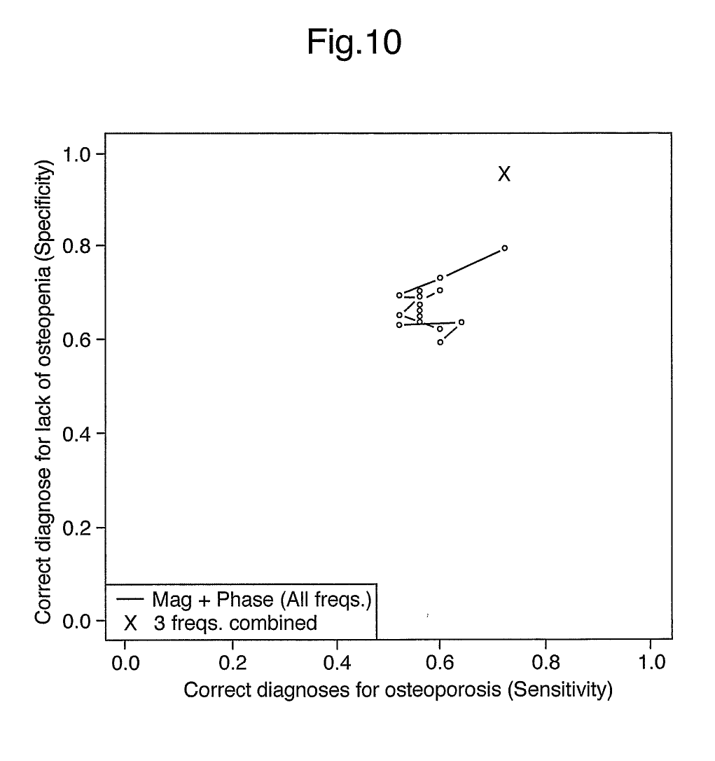

Analysis of impedance measurements against BMD values from DEXA using suitable statistical methods also demonstrate the applicability of this method. FIGS. 9 and 10 show that by using Fisher Linear Discrimination Analysis a classifier can be generated to yield sensitivity and specificity of at least 80%. By further analysing suitable data sets, it is possible to generate classifiers that achieve at least 90% sensitivity and specificity.

It will be appreciated therefore that the electrical impedance characteristics can clearly be used to correlate to the structure of the bone tissue, in this case in the form of BMD values.

It is envisaged that the above method and apparatus may be used as an effective screening device for osteoporosis and/or osteopenia and in a long term monitoring program whereby subjects are monitored repeatedly over a period of time. A typical period could be five years between measurements. These could begin in early adulthood for women in particular and the results could be used after each five year period to compare with “T” or “Z” values relating to representative average values for similar healthy members of the adult population according to their age. Similarly for particular subjects with medical conditions the latter could be used to show trends with respect to other such subjects with similar conditions so as to monitor the progression or regression of osteopenia or osteoporosis. In addition, such control groups could also be subdivided according to their weight and other body characteristics. If as a result of the screening program any particular subject is found to be deteriorating at a rate which is not acceptable medically, then a treatment program could be prescribed including one or more of a change in lifestyle, diet, or the use of drugs to arrest the condition.

It is also noted here that osteopenia and osteoporosis may also be caused by the use of certain drugs for the treatment of other conditions and the abovementioned screening or monitoring method may be used to assess the side effects of the use of such drugs.

It will be appreciated that the apparatus described can take a number of forms, including a handheld device, a portable device or indeed a static device. It is not essential that each of the components described within the unit 2 is positioned within a single unit and indeed it is envisaged that a more portable device may be provided for applying the signals and monitoring the response of the circuit, whereas the computing display and input devices may be positioned in a separate device which communicates directionally with such a handheld device via either a wired or wireless link (such as a Bluetooth link).

The example described uses a two electrode system and it is being found by the present inventors that a two electrode arrangement provides excellent results and ease of use. Whilst in principle it is possible to use a four electrode measurement system, in which two electrodes are used to apply the signals and a further two electrodes are used to monitor the response of the circuit, practically these add to the complexity and cost of such apparatus and also reduce its ease of use. The present apparatus is envisaged as being useful in a medical context for over-the-counter sales to members of the public or at point-of-care in local “general practitioner surgeries”, and also to hospitals. Thus the apparatus provide an extremely low cost alternative to the expensive DEXA system while avoiding the use of harmful ionizing X-ray radiation.

1-35. (canceled)

36. A method of analysing the structure of bone tissue comprising:
a) placing first and second electrodes in electrical contact with the bone tissue to be analysed such that the bone tissue forms at least part of an electrical circuit between the first and second electrodes;
b) applying an alternating electrical signal to the circuit and monitoring the electrical response of the circuit; and,
c) processing the monitored response to generate output data representative of the structure of the bone tissue.

37. A method according to claim 36, wherein in step (b) the first and second electrodes are used to apply the current to the circuit and to monitor the response.

38. A method according to claim 36, wherein the alternating current is applied at multiple frequencies.

39. A method according to claim 38, wherein the alternating current is applied at different frequencies in a serial manner.

40. A method according to claim 38, wherein the alternating current is applied at different frequencies in a simultaneous manner.

41. A method according to claim 36, wherein one or more frequencies of the alternating current is less than 20 kHz.

42. A method according to claim 41, wherein one or more frequencies of the alternating current is less than 10 kHz.

43. A method according to claim 42, wherein one or more frequencies of the alternating current is less than 1 kHz.

44. A method according to claim 43, wherein one or more frequencies of the alternating current is 200 Hz or less.

45. A method according to claim 36, wherein the method is performed in vivo.

46. A method according to claim 45, wherein the first electrode is placed at or adjacent a first surface of the bone tissue and the second electrode is placed at or adjacent a second surface of the bone tissue.

47. A method according to claim 46, wherein one or each of the first and second electrodes is placed against the skin or a subject body containing the bone tissue.

48. A method according to claim 46, further comprising applying an electrically conductive material to each electrode and/or the surface against which the electrode is placed.

49. A method according to claim 36, wherein the processing of the response comprises deriving one or more electrical characteristics from the monitored response and comparing the said one or more characteristics with a structure characteristic of the bone tissue according to a predetermined relationship.

50. A method according to claim 49, wherein the one or more electrical characteristics includes one or more of the current response, voltage response and the phase shift between the applied current and the voltage response.

51. A method according to claim 49, wherein the predetermined relationship includes a combination of the electrical characteristics.

52. A method according to claim 49, wherein the structure characteristic is a measure of bone density.

53. A method according to claim 36, wherein the applied current in the circuit is less than 100 microamperes.

54. A method according to claim 36, wherein the first and second electrodes are positioned in a hip region, heel region, spine region or forearm region of a subject body.

55. A method according to claim 54, wherein when the bone tissue of a subject forearm is to be analysed then one of the first and second electrodes is positioned adjacent the styloid process of the radius or ulna and the other of the first and second electrodes is positioned adjacent the olecranon process.

56. Apparatus for analyzing the structure of bone tissue comprising:
a first and second electrode in electrical contact with the bone tissue to be analysed such that the bone tissue forms at least part of an electrical circuit between the first and second electrodes;
a signal generator adapted to apply an alternating electrical signal to the circuit through the first and second electrodes;
a monitoring device for monitoring the electrical response of the circuit; and,
a processor adapted when in use to process the response monitored by the monitoring device and to generate output data representative of the structure of the bone tissue.

57. Apparatus according to claim 56, wherein the signal generator is adapted to generate electrical signals at a plurality of frequencies.

58. Apparatus according to claim 57, wherein the signal generator is adapted to provide the plurality of frequencies in a serial manner.

59. Apparatus according to claim 56, wherein the signal generator is adapted to generate electrical signals having a frequency of less than 20 kHz.

60. Apparatus according to claim 59, wherein the signal generator is adapted to generate electrical signals having a frequency of less than 10 kHz.

61. Apparatus according to claim 60, wherein the signal generator is adapted to generate electrical signals having a frequency of less than 1 kHz.

62. Apparatus according to claim 61, wherein the signal generator is adapted to generate electrical signals having a frequency of 200 Hz or less.

63. Apparatus according to claim 56, wherein the signal generator and the monitoring device are formed within a single unit.

64. Apparatus according to claim 56, wherein the monitoring device is adapted to monitor the electrical response of the circuit through the first and 15 second electrodes.

65. Apparatus according to claim 56, wherein one of the first or second electrodes is formed having an electrically conductive contact surface containing a depression suitable to receive the elbow of a human subject.

66. A method according to claim 36, wherein the indicated structure is an osteoporotic or osteopoenic bone tissue structure.

67. A method of monitoring changes in the structure of bone tissue within a subject body, comprising:
analysing the structure of the bone tissue of a particular subject at different times using a method according to claim 36;
comparing the bone structure represented by the output data with bone structure representative of the type of subject monitored; and,
selectively applying a treatment to subject body as a result of the comparison.

68. A method according to claim 67, wherein the analysis times are separated by at least one year.

69. A method according to claim 67, wherein the type of subject includes one or more of the sex, age, size, weight and medical conditions.

70. A method according to claim 67, wherein the treatment is applied if the analysed subject has an indicated bone structure which differs from the representative structure by more than a predetermined amount.
